(19) United States
(12) Patent Application Publication
(10) Pub. No.: US 2023/0316141 A1
(43) Pub. Date: Oct. 5, 2023

(54) SYSTEMS AND METHODS FOR WEIGHTED FEDERATED LEARNING IN A HYBRID OPERATING ROOM ENVIRONMENT

(71) Applicant: KONINKLIJKE PHILIPS N.V., EINDHOVEN (NL)

(72) Inventors: GRZEGORZ ANDRZEJ TOPOREK, CAMBRIDGE, MA (US); AYUSHI SINHA, BALTIMORE, MD (US); LEILI SALEHI, WALTHAM, MA (US); RAMON QUIDO ERKAMP, SWAMPSCOTT, MA (US); ASHISH SATTYAVRAT PANSE, BURLINGTON, MA (US)

(21) Appl. No.: 18/127,272

(22) Filed: Mar. 28, 2023

Related U.S. Application Data
(60) Provisional application No. 63/325,787, filed on Mar. 31, 2022.

(51) Int. Cl. G06N 20/00; G16H 40/20

(52) U.S. Cl. CPC G06N 20/00; G16H 40/20

(57) ABSTRACT

The present disclosure relates to a method for a federated learning system in hybrid operating room environment, in which a global machine-learning model learns from a subset of local participants selected based on predefined selection criteria. The local participants amongst the subset respectively provide an update matrix and a quality score to train the global machine-learning model. A combination of the update matrix and the quality score is applied to the global machine-learning model, in which each update matrix is weighed based on the quality score of the local participant.

### CROSS REFERENCE TO RELATED APPLICATION

This application claims the benefit of U.S. Provisional patent application Ser. No. 63/325,787, filed Mar. 31, 2022. This application is incorporated by reference herein.

### FIELD OF THE DISCLOSURE

The present disclosure relates to machine-learning and predictive analytics, and more specifically, to computer-implemented systems and methods for weighted federated learning of medical images and modeled parameters in a hybrid operating room environment.

### BACKGROUND

Conventionally, federated learning, also referred to as collaborative learning, indicates a decentralized machine-learning technique in which local computing devices collectively train a shared machine-learning model by use of respective local datasets without exchanging the local dataset with other computing devices. However, one of the challenges of federated learning systems is statistical heterogeneity. For example, in the medical or hospital setting, local datasets generated and collected by client devices may be non-identically distributed, e.g. procedures from some clients may involve only certain patients, or another set of clients may be less experienced with a certain types of procedures. Moreover, the number of training instances across different clinical sites may vary significantly, and there might be underlying biases in the data, e.g. only certain subjects, or an area subjected to a specific condition. Dissimilar data distributions violate frequently used independent and identically distributed assumptions of distributed optimization in federated learning systems thus leading to poor performance of machine-learned models.

### SUMMARY OF THE DISCLOSURE

The present disclosure is directed to systems and methods for federated learning amongst a global server and a plurality of local participants in a hybrid operating room environment. The systems and methods keep local image data in the local dataset to protect patient privacy but facilitate local analysis of the local image data to be analyzed consistently throughout the federated learning system and coordinate analyses by the local participants by analyzing statistics of the image data on the global server. The systems and methods of the present disclosure improve techniques for federated learning by accounting for statistical heterogeneity and biases in the global model inherent in conventional federated learning.

In accordance with aspects of the present disclosure, a computer-implemented method includes: selecting, by one or more processors, a subset from a plurality of local participants each associated with a hybrid operating room and subscribing to a federated learning system to contribute to a global machine-learning model based on predefined selection criteria on the local participants for the subset; obtaining, by the one or more processors, an update matrix and a quality score from each local participant in the subset from the local participants; updating, by the one or more processors, the global machine-learning model with the update matrix from respective to each local participant in the subset as being weighed based on the quality score; and deploying, by the one or more processors, parameters of the global machine-learning model and weights corresponding to the parameters as being updated to the local participants in the federated learning system for respective local learning.

In an aspect, the method also includes: ascertaining that the global machine-learning model as being updated needs to be validated prior to a deployment to the local participants in the federated learning system; and validating that the global machine-learning model as being updated performs better than a version of the global machine-learning model prior to being updated, by use of a validation dataset stored in a global server from which the global machine-learning model is trained.

In an aspect, the method also includes: the predefined selection criteria including: an availability schedule for each of the local participants in the federated learning system, the quality score for each of the local participants, a mode of subscription to the federated learning system by each of the local participants, and a quality of data generated from each of the local participants.

In an aspect, the method also includes: in the updating, aggregating weights in the update matrix from each local participants based on the quality score into aggregate weights for respective parameters in the update matrix; and incorporating the aggregated weights for the respective parameters in the update matrix into respective weights for parameters of the global machine-learning model corresponding to the respective parameters of the update matrix.

In an aspect, the method also includes: in the deploying, sending parameters of the global machine-learning model as being updated based on the update matrix and the quality score respective to each local participants in the subset to the local participants in the federated learning system.

In an aspect, the method also includes: facilitating one or more local learning tools to be installed in or downloaded to the local participants in the federated learning system, the local learning tools for analyzing and evaluating data stored on respective local participants consistently across the local participants in the federated learning system.

In an aspect, the method also includes: obtaining local statistics on predetermined aspects of each local participants in the subset; analyzing correlations between the predetermined aspects and a task of the global machine-learning model as represented by differences in the local statistics and global statistics on the predetermined aspects from the local participants in the federated learning system; and adjusting weights of parameters in the global machine-learning model based on the correlations.

In an aspect, the method also includes: the global machine-learning model includes weighting of local model parameter contributions to global model parameters according to one or more: institute criteria on organizational characteristics of the local participants; imaging system criteria based on technical specifications of imaging systems in hybrid operating rooms of the local participants; procedure criteria based on characteristics of procedures performed in the hybrid operating rooms; healthcare processional criteria based on characteristics of medical personnel working for the local participants; and patient criteria based on characteristics of patients receiving the procedures performed in the hybrid operating rooms.

In an aspect, the method also includes: the predefined selection criteria for the subset comprise availability of the local participant, the quality score of the local participant, a mode of subscription of the federated learning system by the local participant, and a quality of data generated from the local participant.

In accordance with aspects of the present disclosure, a computer-implemented method includes: building, by one or more processors of a local participant, a local machine-learning model based on parameters of a global machine-learning model and respectively corresponding weights received from a global server of a federated learning system to which the local participant subscribes, wherein the local participant is associated with a hybrid operating room for surgical procedures assisted by medical imaging equipment generating image data in a local dataset of the local participant; training, by the one or more processors, the local machine-learning model with selected instances from the local dataset of the local participant and local statistics; generating, by the one or more processors, an update matrix representing a collection of respective differences in weights between respective parameters of the local machine-learning model before and after a learning cycle based on the local dataset; sending, by the one or more processors, the update matrix, a quality score of the local participant, and the local statistics to the global server; and receiving, by the one or more processors, the parameters of the global machine-learning model and the weights respectively corresponding to the parameters from the global server, as being updated based on the update matrix, the quality score, and the local statistics of the local participant, sent from all local participants in the federated learning system.

In an aspect, the method also includes: computing the quality score of the local participant, the quality score indicating a level of contribution to the global machine-learning model intended by the local participant based on the local dataset.

In an aspect, the method also includes: the local statistics stored in the local dataset comprises historical data generated from or observed at the local participant.

In an aspect, the method also includes: downloading one or more local learning tools from the global server of the federated learning system; analyzing image data stored in the local dataset of the local participant by use of the local learning tools; and sending statistics resulting from the analyzing the image data to the global server with weights respective to features from the image data as parametrized in the local machine-learning model, the weights being calculated based on respective contribution of parametrized features from the image data to a task set to be performed by the local machine-learning model.

In accordance with aspects of the present disclosure, a system includes: a memory, one or more processors in communication with the memory, and program instructions executable by the one or more processors via the memory configured to: select a subset from a plurality of local participants each associated with a hybrid operating room and subscribing to a federated learning system to contribute to a global machine learning model based on predefined selection criteria on the local participants for the subset; obtain an update matrix and a quality score from each local participant in the subset from the local participants; update the global machine learning model with the update matrix from respective to each local participant in the subset as being weighed based on the quality score; and deploy parameters of the global machine learning model and weights corresponding to the parameters as being updated to the local participants in the federated learning system for respective local learning.

In an aspect, the system is also configured to: ascertain that the global machine learning model as being updated needs to be validated prior to a deployment to the local participants in the federated learning system; and validate that the global machine learning model as being updated performs better than a version of the global machine learning model prior to being updated, by use of a validation dataset stored in a global server from which the global machine learning model is trained.

In accordance with aspects of the present disclosure, a system for machine-learning in a hybrid operating room environment includes a global server comprising one or more processors configured to: select a local participant subset from a plurality of local participants associated with a hybrid operating room; subscribe the local participant subset to a federated learning system to contribute to a global machine-learning model configured to perform a task in the hybrid operating room; obtain an update matrix and a quality score from each local participant in the local participant subset; update the global machine-learning model based on the update matrix and the quality score from each local participant in the local participant subset; and deploy parameters and parameter weights of the global machine-learning model to each local participant in the local participant subset for building a local machine-learning model configured to perform the task in the hybrid operating room.

In an aspect, the one or more processors of the system are also configured to: ascertain that the updated global machine-learning model needs to be validated prior to the deployment to each local participant in the federated learning system; and validate that the updated global machine-learning model performs better than a pre-updated version of the global machine-learning model, by use of a validation dataset stored in the global server from which the global machine-learning model is trained. In an aspect, the system includes a local participant subset selected based on a predefined selection criteria comprising at least one of: an availability schedule for each local participant in the federated learning system, the quality score for each local participant in the federated learning system, a mode of subscription to the federated learning system by each local participant in the federated learning system, and a quality of data generated from each local participant in the federated learning system.

In an aspect, the one or more processors of the system are also configured to: aggregate weights of parameters in the update matrix from each local participant based on the respective quality score from each local participant; and incorporate the aggregated weights of the parameters in the update matrix into parameter weights of corresponding parameters in the global machine-learning model. In an aspect, the one or more processors of the system are also configured to: send the parameters of the global machine-learning model to each local participant in the federated learning system. In an aspect, the one or more processors of the system are also configured to: obtain local statistics on predetermined aspects of each local participant in the local participant subset; analyze correlations between the predetermined aspects and a task of the global machine-learning model as represented by differences in the local statistics and global statistics on the predetermined aspects in the federated learning system; and adjust the parameter weights of the parameters in the global machine-learning model based on the correlations.

In an aspect, the global machine-learning model of the system comprises weighting of local machine-learning model parameter contributions to the parameters of the global machine-learning model according to one or more of: institute criteria on organizational characteristics of each local participant; imaging system criteria based on technical specifications of imaging systems in the hybrid operating rooms of each local participant; procedure criteria based on characteristics of procedures performed in the hybrid operating rooms; healthcare processional criteria based on characteristics of medical personnel working for each local participant; and patient criteria based on characteristics of patients receiving the procedures performed in the hybrid operating rooms.

In an aspect, the system includes a local client of a local participant in the local participant subset and the local client includes one or more local processors configured to: build the local machine-learning model configured to perform the task in the hybrid operating room of the local participant based on the parameters and the parameter weights of the deployed global machine-learning model, wherein the local participant includes a local dataset with image data and parameters associated with medical imaging equipment of the hybrid operating room of the local participant; train the local machine-learning model with selected instances from the local dataset and local statistics of the local participant; generate an update matrix representing a collection of respective differences in weights between respective parameters of the local machine-learning model before and after a learning cycle based on the local dataset; send, to the global server, the update matrix, the quality score, and the local statistics for the local participant; receive the parameters and the parameter weights of the global machine-learning model updated based on the update matrix, the quality score, and the local statistics sent from each local participant in the federated learning system; and update the local machine-learning model for the local participant based on the updated parameters and the updated parameter weights of the global machine-learning model.

In an aspect, the one or more local processors of the system are further configured to: compute the quality score of the local participant to indicate a level of contribution to the global machine-learning model by the local participant based on the local dataset. In an aspect, the local statistics stored in the local dataset of the system comprises historical data generated from or observed at the local participant. In an aspect, the one or more local processors of the system are further configured to: download one or more local learning tools from the global server; analyze image data stored in the local dataset of the local participant by use of the one or more local learning tools; and send statistics resulting from the analyzed image data to the global server with weights of features, from the image data, parametrized in the local machine-learning model, wherein the weights of the features are calculated based on contribution of the parametrized features from the image data to a task set configured to be performed by the local machine-learning model.

It will be appreciated by those skilled in the art that two or more of the above-mentioned embodiments, implementations, and/or optional aspects of the present disclosure may be combined in any way deemed useful. Modifications and variations of any system and/or any computer readable medium, which correspond to the described modifications and variations of a corresponding computer-implemented method, can be carried out by a person skilled in the art on the basis of the present description.

### BRIEF DESCRIPTION OF THE DRAWINGS

These and other aspects of the present disclosure will be apparent from and elucidated further with reference to the embodiments described by way of example in the following description and with reference to the accompanying drawings, in which:

FIG. 1 shows functional components of a federated learning system;

FIG. 2 shows a flowchart describing a computer-implemented method performed by the federated learning system of FIG. 1;

FIG. 3 depicts a detailed workflow of the global server in a round of training for the global model;

FIG. 4 depicts a detailed workflow of a local trainer of the local participant in a round of training for the local model;

FIG. 5 illustrates exemplary graphs from the local statistics and the global statistics on a few parameters of the global model for predicting a duration of navigation to a procedure site in mechanical thrombectomy; and

FIG. 6 shows a computer-readable medium comprising data.

It should be noted that the figures are purely diagrammatic and not drawn to scale. In the figures, elements which correspond to elements already described may have the same reference numerals.

### DETAILED DESCRIPTION OF EMBODIMENTS

The present disclosure is directed to systems and methods for federated learning amongst a global server and a plurality of local participants in a hybrid operating room environment. The systems and methods keep local image data in the local dataset to protect patient privacy but facilitate local analysis of the local image data to be analyzed consistently throughout the federated learning system and coordinate analyses by the local participants by analyzing statistics of the image data on the global server. The systems and methods of the present disclosure improve techniques for federated learning by accounting for statistical heterogeneity and biases in the global model inherent in conventional federated learning. The systems and methods of the present disclosure can be used to train a machine-learned model for any type of task, e.g., classification, segmentation, regression specific to a sensory data provided by a medical device (e.g., x-ray system, laser atherectomy device, stent or balloon with sensing capabilities) deployed during a medical procedure, such as, for example, minimally invasive biopsy, percutaneous coronary intervention (PCI) procedures, spinal fusion procedures, or orthopaedic surgery.

Conventionally, federated learning, also referred to as collaborative learning, indicates a decentralized machine-learning technique in which local computing devices collectively train a shared machine-learning model by use of respective local datasets without exchanging the local dataset with other computing devices. In relation with federated learning, edge computing refers to any distributed processing model on various tasks across geographically dispersed computing devices, often in cloud computing environment. In federated learning, a federated server performs a certain level of coordination for all participants in the federated learning. With federated learning, a machine-learning model can be trained globally on real world data without centrally storing a global dataset or moving local dataset to other participants of the federated learning. Accordingly, federated learning is a proper way to utilize sensitive data for globally training machine-learning models without moving such sensitive data.

Hybrid operating rooms are surgical operating rooms that are equipped to enable diagnostic imaging before, during, and after surgical procedures. Imaging during surgical procedures is also referred to as interventional imaging. Medical imaging equipment is installed in the hybrid operating room. As hybrid operating rooms equipped with medical imaging devices will generate sensitive data that must not be moved to other sites as governed by privacy regulations, federated learning would be suitable for training a predictive analytical model for data collected from such medical image data from hybrid operating rooms, to obtain insights and further knowledge on complex medical procedures performed in such hybrid operating rooms and preconfigured features thereof.

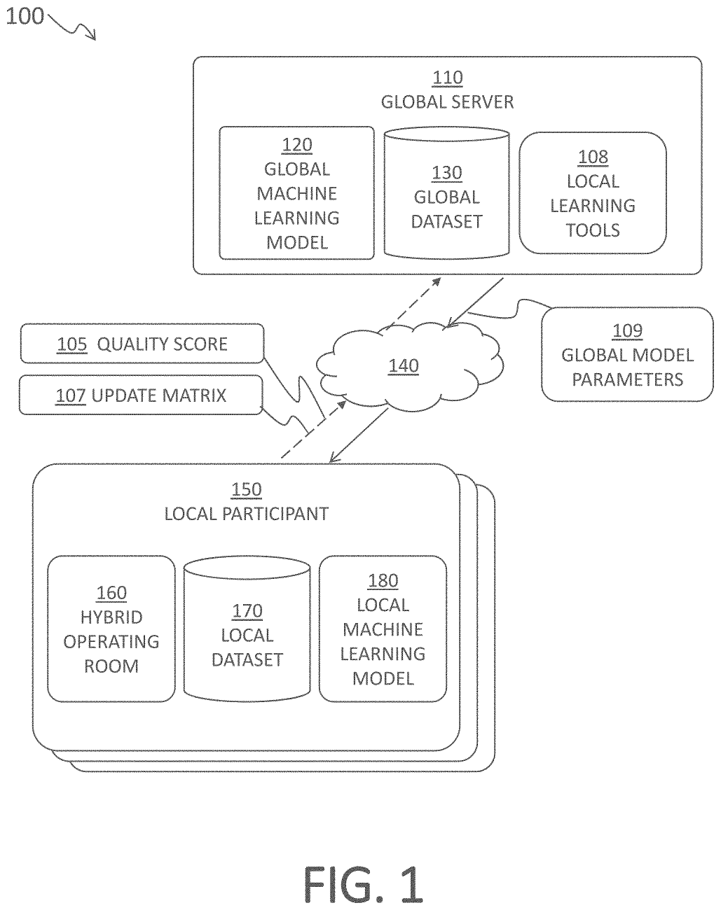

FIG. 1 shows functional components of a federated learning system 100.

The federated learning system 100 includes a global server 110 and a plurality of local participants associated with respective hybrid operating rooms, represented by a local participant 150. The global server 110 and the local participant 150 include respective computing platforms operatively coupled to a digital communication network 140. The computing platform(s) of the local participant 150 includes one or more graphics processing units (GPU) for image data of the local participant 150.

The global server 110 includes a global machine-learning model 120, referred to as a global model 120, and a global dataset 130. The global model 120 may also be referred to as central model, shared model, combined model, and so on. The global dataset 130 includes global statistics and expert annotations but does not store any image data from the local participants 150. The global server 110 sends the local participants 150 a plurality of global model parameters 109 of the global model 120 as trained based on the global dataset 130. The global server 110 also includes local learning tools 108 which would be sent to the local participants 150 in executable forms.

In certain embodiments, the global server 110 can include a collection of regional servers that correspond to respective geographical areas covering a certain number of local participants such that the global server 110 coordinates only aspects that affects all local participants across the federated learning system 100 in its entirety but the respective regional servers would coordinate aspects that affect a specific region based on demographics or medical infrastructure of the specific region. In this disclosure, the global server 110 represents any types of servers in the federated learning system 100 that coordinates local learnings by the local participants 150.

In an example, the local participant 150 includes at least one hybrid operating room (OR) 160, a local dataset 170, and a local machine-learning model 180, referred to as a local model 180. The local participant 150 receives the global model parameters 109 of the global model 120 from the global server 110 and sends an update matrix 107, a quality score 105, and local statistics to the global server 110. The local participant 150 can have the local learning tools 108 downloaded from the global server 110 upon request such that the local participant 150 can perform image analysis and evaluation offered by the local learning tools 108 on the local dataset 180 in training the local model 170, without sending sensitive data stored in the local dataset 180 to the global server 110. The local dataset 180 is not limited to data collected at the local participant 150, but may include any data available to the local participant 150 through data collected in a hybrid OR environment, data collected through study participation at a different site but available to the local participant 150 to access, data collected through procedures performed remotely by remote operators of procedure equipment, and so on. Examples of the local learning tools 108 include, but are not limited to, a tortuosity detector, an image quality (IQ) processor application, a plaque detector, or other medical image processing tools.

The hybrid operating room 160 of the local participant 150 indicates a room in which surgical or interventional procedures would be performed and includes one or more medical/interventional imaging equipment, including mobile components, permanently installed for such procedures. Examples of the medical imaging equipment in the hybrid operating room 160 include, but are not limited to, a fixed x-ray system, a C-arm x-ray system, or any other type of medical imaging equipment for fluoroscopy to obtain real-time moving images or waveform data of an interior of a surgery site on a patient. Examples of the surgical procedures often performed in the hybrid operating room 160 are often endovascular procedures and complex surgeries in which real time monitoring of the surgery site is critical for the safety of patients. Exemplary types of images generated by the imaging equipment in the hybrid operating room 160 or other healthcare environments include, but are not limited to, computed tomography (CT) scans, magnetic resonance imaging (Mill) scans, single photon emission computed tomography (SPECT) scans, cone beam CT scans, computed tomography angiography (CTA) scans, X-ray images, fluoroscopy scans, digitally subtracted angiography (DSA) sequences as processed by guidance applications for surgical devices accompanying the imaging equipment.

The local dataset 170 includes image data generated from the hybrid OR 160 that may need image analysis technology for processing not available from the local participant 150. Due to privacy regulations, however, the local dataset 170 cannot be sent to the global server 110. Accordingly, in the federated learning system 100, the local participant 150 can request the image analysis tools 108 from the global server 110 to analyze the image data in the local dataset 170 without moving data to the global server 110. The local dataset 170 further includes local statistics on various aspects of the local participant 150, in addition to specified types of medical data.

The local participant 150 builds the local model 180 based on the global model parameters 109 previously received from the global server 110 and local configurations specifically set for the local participant 150. The local participant 150 trains the local model 180 with the local dataset 170, including image data generated from the hybrid operating room 160 of the local participant 150. The local dataset 170 is stored locally or at a secure site according to any applicable medical data privacy laws and regulations and will not be transmitted to the global server 110, which would significantly reduce the volume of data traffic between the local participant 150 and the global server 110 as well as protect sensitive medical data of patients who had used the hybrid operating room 160.

The update matrix 107 of the local participant 150 indicates a collection of differences in weights between respective parameters of the local model 180 before and after a learning cycle based on the local dataset 170. In the federated learning system 100, the local participant 150 participates in the federated learning system 100 by generating the update matrix 107 based on training the local model 180 with the local dataset 170 in comparison with the global model parameters 109 and by sharing the update matrix 107 with the global server 110, without sharing content of the local dataset 170.

The quality score 105 of the local participant 150 indicates a level of contribution to the global model 120 by the local participant 150, that is, how significant a role of the local participant 150 would be within the federated learning system 100. The quality score 105 is computed based on the number of criteria, optionally grouped for classification, obtained from each local participant. For example, the quality score 105 of the local participant 150 can be derived automatically from a certain x-ray system, any medical equipment, sensory data, as generated from the hybrid OR 160 or institutional characteristics of the local participant 150 or extracted using the local learning tools 108. The local participant 150 calculates the weights for parameters in the update matrix 107 based on the quality score 105.

The local participant 150 can also provide the quality score 105 directly to the global server 110 such that the global server 110 can incorporate the update matrix 107 to the global model 120 according to the quality score 105 of the local participant 150. The quality score 105 of the local participant 150 can change over time based on changes in the size and quality of operations of the local participant 150 such as imaging equipment in the hybrid OR 160, the number of operations performed in the hybrid OR 160, qualifications, specialization, and/or reputation of medical personnel of the local participant 150, new discoveries in medical knowledge and invention of new procedures, and patient demographics. Detailed lists of exemplary attributes in the criteria affecting the quality score 105 of the local participant 150 are presented below.

In one of learning cycles of the federated learning system 100, the local participant 150 generates the update matrix 107 based on training the local model 180 with selected part of the local dataset 170 and sends the update matrix 107 to the global server 110, as well as local statistics. Upon receiving the update matrix 107 from the local participant 150, the global server 110 updates the global model 120 with a current batch of update matrices from selected local participants, including the update matrix 107 when the local participant 150 is selected, and sends the global model parameters 109 of the global model 120 as being updated and/or validated, upon deploying the global model 120. The local participant 150 corresponds to the quality score 105 as noted above. The quality score 105 indicates a level of contribution from the local participant 150 to the global model 120 and the global dataset 130, that is, how significantly the global server 110 would take account of the update matrix 107 from the local participant 150 in updating the global model 120.

In certain embodiments, the global model 120 may be further updated or retrained on an expanded global dataset 130 whenever additional data, annotations, or global statistics are available to the global server 110. The global model 120 may be initialized to its most recent parameters and the parameters may be updated by retraining on the expanded global dataset 130 directly before deploying the updated global model parameters 109 to the local participants 150. Alternatively, the global model parameters 109 may be updated by training on the expanded global dataset in combination with the most recent update matrix 107 available from local participants 150, for instance, by weighting the updated global parameters 109 by the update matrix 107 weighted by the quality score 105 of the local participants 150 after each training iteration.

The global model 120 is one or more machine-learning models on the global server 110 that would be deployed to the local participants 150 with the global model parameters 109. The global model 120 represents a model trained using currently available machine-learning techniques including, but not limited to, convolutional neural networks (CNN), multi-layer perceptron (MLP), recurrent neural networks (RNN) and long short-term memory (LSTM) architecture, generative adversarial networks (GAN), vanilla or variational encoder-decoder networks (VAE), transformer networks, support vector machine (SVM), decision tree or random forest models, multivariate regression including weighted linear or logistic regression.

In certain embodiments, the global model 110 utilizes stochastic gradient descent (SGD) for optimization in training the global model 120. Examples of optimization techniques that can be used by the global server 110 in training the global model 120 include mini-batch gradient descent, the Broyden—Fletcher—Goldfarb—Shanno (BFGS) algorithm, limited memory-BFGS (L-BFGS), adaptive moment estimation (ADAM), nonlinear conjugate gradient, Levenberg-Marquardt algorithm (LMA or LM). Examples of loss functions include cross-entropy loss for classification tasks, binary cross entropy, mean squared error (MSE), mean absolute error (MAE), structural similarity index measure (SSIM), least absolute deviations (L1), least square errors (L2 or LS), discriminator or perceptual loss in generative adversarial network (GAN), and Kullback—Leibler (KL) divergence in variational autoencoders.

Model parameters as described with reference to global model parameters 109 and local model parameters may include parameters that are optimized during each iteration of model training, including weights, w, and biases, b, hyperparameters that are optimized, for instance, using a held out validation dataset, including learning rate, regularization methods, weight decay and augmentation methods, and any other parameters that introduce a change in the model and, therefore, a change in the outputs of the model, including model architecture and number of neural network layers.

The global dataset 130 indicates a large set of data that the global server 110 has access to within various privacy policies, that is stored on the global server 110, along with global statistics derived from the global dataset 130. The global dataset 130 is used to initially train the global model 120 prior to deployment to the local participants 150. The global dataset 130 can be restricted with access from the local participants 150 and labelled and processed to prevent any statistical biases common in conventional federated learning system. The global server 110 can update the global dataset 130 with additional data obtained within data privacy policies and/or with annotations by human experts or expert systems on matters subject to federated learning. The global dataset 130 can include a held out validation dataset to screen update matrices from the local participants or to validate performance of the global model 120 after training.

As noted above, federated learning on medical images and statistical data generated from hybrid ORs 160 can advance medical knowledge and insights on procedures performed in hybrid ORs 160 and generate high quality predictions for various procedures and patients. Medical data are mostly subject to strict privacy protection by rules and regulations, which require such medical data to remain local and not to transfer without informed consent from patient. Accordingly, federated learning as implemented in the federated learning system 100 is a suitable method for collaboration in cumulating the knowledge while protecting patient privacy. Further, medical images and other information and statistics separated from patient identity information can often be used and shared for research purposes as being authorized by patients. The federated learning system 100 facilitates collaborative learning by the global server 110 and private learning by respective local participants 150 as separating the local dataset 170 from the global dataset 130. Accordingly, the federated learning system 100 can also dramatically reduce data traffic necessary for the collaborative learning between the local participant 150 and the global server 110. For certain local participants 150 that generate significant amount of data of high quality in comparison with other local participants can be benefited from the contribution to the federated learning system 100 by, for example, directly monetizing updates to the global server 110 or advertising a good reputation for such contribution to the public such that potential patients would recognize the local participant 150 as an expert to a procedure from which the updates were generated. Also, because the updates from the local participants 150 are based on real procedures, the field of medical knowledge and the quality of care regarding the procedure of interest can be advanced and improved with the updates from the local participants 150.

In conventional federated learning mechanisms, however, respective participants may generate statistically heterogeneous data based on characteristics of operations or procedures by the respective participants such that a certain group of participants may not be benefitted from contributions to a federated model by another group of participants in a same collaborative learning infrastructure. Further, sizes, types, and qualities of training datasets respective to participants can be biased according to, for example, types of medicine practiced by the respective participants, diagnoses that affect patients from a particular demographic, and respective status of public health and demography in locations and/or regions in which the respective participants operate. Such statistical heterogeneity and biases in datasets respective to the participants may result in degraded performance in a resulting federated model as federated learning assumes that contributions to the federated learning model from respective participants would be independent and identically distributed (iid). In order to accommodate the statistical heterogeneity and biases amongst the participants, certain conventional federated learning infrastructures may use a quality score to assess the respective relevance of each participant in generating ground-truth data such that only participants with the quality score greater than a certain threshold value can contribute to retraining of a federated model and/or updating certain parameters of the federated model, or the retraining and/or update from various participants can be weighted accordingly by the quality scores.

The federated learning system 100 of the present disclosure addresses the statistical heterogeneity and biases in conventional federated learning by applying respective quality scores corresponding to the local participants 150 in the process of taking each of update matrices 107 from the local participant 150 into account for the global model 120 in the global server 100. The quality score 105 corresponding to the local participant 150 can be assessed, by the local participant 150 or by the global server 110. The update matrix 107 of the local participant 150 is then reflected in the global dataset 130 and used in retraining of the global model 120 for the local participant 150 with the quality score 105 greater than other local participants. The global model parameters 109 may be updated by local model parameter updates weighted by attributes related to technical data and medical records, including but not limited to attributes specific to imaging technology and equipment, steps of interventional procedures from which images are generated and results therefrom, and attributes of patients undergoing the procedure. Attributes may further be related to comprehensive operational information of the local participant 150 as a medical institute, and medical personnel of the local participants 150 who would be performing the procedures in the hybrid operating room 160. Accordingly, the global model 120 may make more accurate predictions than conventional federated learning based on local model parameters weighted according to criteria covering extensive data points in all aspects of the procedures performed in the hybrid operating room 160, and that the local participants 150 are associated with respective quality scores representing a contributory value of the local dataset 170 and the local model 180, and accordingly, indicating a how the respective update matrices 107 should be handled in training of the global model 120.

In certain embodiments, the global model parameters 109 may be updated by local model parameter updates weighted according to one or more criteria, for example, institute criteria based on organizational characteristics of the local participants 150, imaging system criteria based on technical specifications of the hybrid operating room 160 and imaging equipment therein, procedure criteria based on characteristics of procedures performed in the hybrid operating room 160, healthcare professional (HCP) criteria based on characteristics of medical personnel of the local participant 150, and patient criteria based on characteristics of patients receiving the procedure in the hybrid ORs 160

In the same embodiments as above, exemplary attributes in the institute criteria as being included in the update of the global model parameters 109 by weighted local model parameters, and as deployed to the local participant 150, include, but are not limited to: a number of post-procedure complications at the local participant 150; a number of total procedures performed at the local participant 150 per department; a number of patients at the local participant 150 including a number of outpatient visits and hospitalizations; an average cost of procedures per procedure type; a geographical location of the local participant 150 with respect to applicable laws and regulations regarding aspects of the procedure, particularly radiation dose, of the local participant 150; and health-related demographics in the geographical location as being serviced by the local participant 150.

In the same embodiments as above, exemplary attributes in the institute criteria as being included in the update of the global model parameters 109 by weighted local model parameters, and as deployed to the local participant 150, include, but are not limited to: a type of the imaging equipment in the hybrid operating room 160 and enabled applications such as Philips EchoNavigator for fusing echocardiography and X-ray images, Philips VesselNavigator for 3D image fusion tool specialized for endovascular procedures, Philips Fiber Optic RealShape (FORS) 3D device guidance; a quality of image with respect to metrics used for interventional imaging equipment; and the quality of data annotations indicating additional text or graphical marks overlapped on images produced by the imaging equipment in the hybrid operating room 160. (Philips is a registered trademark of Koninklijke Philips N.V., in the United States and other countries.) The data annotations can be provided manually by medical staffs of the local participant 150 or automatically derived from user interactions with the imaging equipment or from other data generating devices in the hybrid operating room 160 such as cameras and other monitoring devices for patients during procedures in the hybrid operating room 160. Exemplary metrics to represent the quality of image include, but are not limited to, signal-to-noise ratio (SNR), contrast, background brightness, viewing angles, prevalence and severity of image artifacts, distortion, and blur of anatomical structures such as blood vessels subject to imaging, classification models.

In the same embodiments as above, exemplary attributes in the institute criteria as being included in the update of the global model parameters 109 by weighted local model parameters, and as deployed to the local participant 150, include, but are not limited to: a number of interactions with the imaging equipment during each procedure including a number of times the imaging equipment is repositioned; a number of fluoroscopy runs; a number of user clicks on user interface of the imaging equipment to obtain a specific view; a number of re-cannulations during each procedure; types and shapes of respective cannulating devices as used during each procedure; the quality of each of the cannulations; a number of maneuvers during each procedure; respective outcomes of the procedures performed at the hybrid operating room 160; viewing angles at the device or anatomical parts on a patient undergoing each procedure; a position of intravenous imaging systems in the hybrid operating room 160 such as intravascular ultrasound (IVUS); tracking accuracy including tracking error such as target registration error (TRE) and fiducial registration error (FRE), shape matching errors as used in Philips FORS, tip location error by electromagnetic tracking; a distance from tracking devices such as optical cameras or electromagnetic field generators to a region subject to imaging for the procedure as influencing accuracies in the image generated; wear-and-tear factor with robotic systems supporting the imaging equipment such as years in use or a number of total procedures performed with the robotics systems; any kinematics calibration errors with the robotics systems; calibration matrices as specified by manufacturers of the imaging equipment, tools used during each procedure such as catheters and guidewires; other tools unsuccessfully attempted during procedure; and details on each procedure such as a level of complexity of a procedure, estimated duration of the procedure, and a likelihood of successful completion of the procedure, and patient profile data in relation with the procedure as derived from an electronic medical record (EMR) for a patient subject to the procedure.

In the same embodiments as above, exemplary attributes in the institute criteria as being included in the update of the global model parameters 109 by weighted local model parameters, and as deployed to the local participant 150, include, but are not limited to: a specialized field of medicine for respective members of the local participant 150 such as board certifications; any medical achievements by respective members of the local participant 150 and public recognition thereof; clinical experiences of respective members of the local participant 150 including years in medical practice and a number of procedures performed; and a number of scientific publications published by respective members of the local participant 150.

In the same embodiments as above, exemplary attributes in the institute criteria as being included in the update of the global model parameters 109 by weighted local model parameters, and as deployed to the local participant 150, include, but are not limited to: basic health indicators including a body mass index (BMI) for respective patients; anatomical abnormalities for respective patients such as tortuous vessels that may affect the procedure of subject to imaging; any existing artificial conditions for respective patients as performed in the past such as a coronary bypass; means of imaging access for respective patients such as a size of acoustic window in ultrasound imaging; and description of a current procedure regarding a complete history of a number of prior procedures, types, or attempts for respective patients such as a first prostatic embolization, a first aneurysm coiling, or a repeat/follow-up procedure.

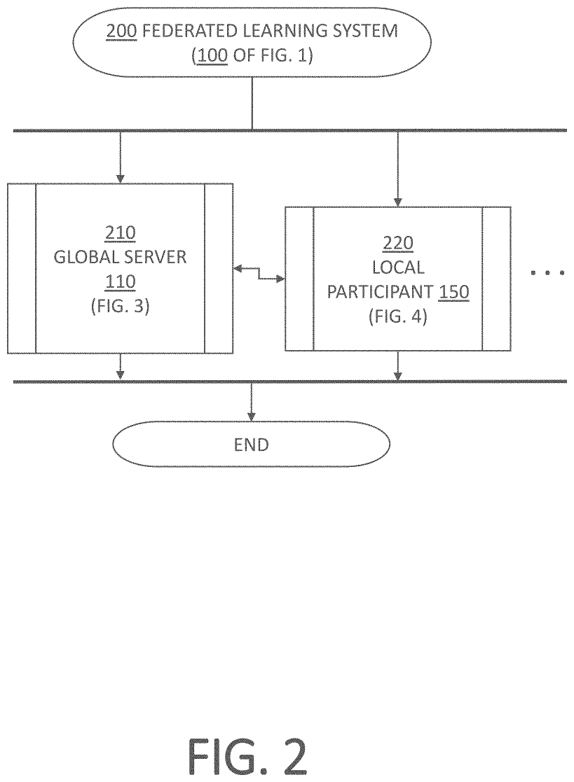

FIG. 2 shows a flowchart 200 describing a computer-implemented method performed by the federated learning system 100 of FIG. 1.

The federated learning system 100 includes the global server 110 and a plurality of local participants 150 respectively interacting with the global server 110, as noted above. The local participant 150 does not interact with other local participants in the federated learning system 100. The global server 110 in block 210 and the local participant 150 in block 220 operate concurrently, run respective learning cycles independently, and exchange the update matrix 107 and the global model parameters 109 as noted herein. It should be noted that the local participant 150 represents each amongst the plurality of the local participants in the federated learning system 100, that many local participants in the federated learning system 100 operate concurrently and independently, and that the global server 210 can communicate with many local participants in the federated learning system 100 simultaneously.

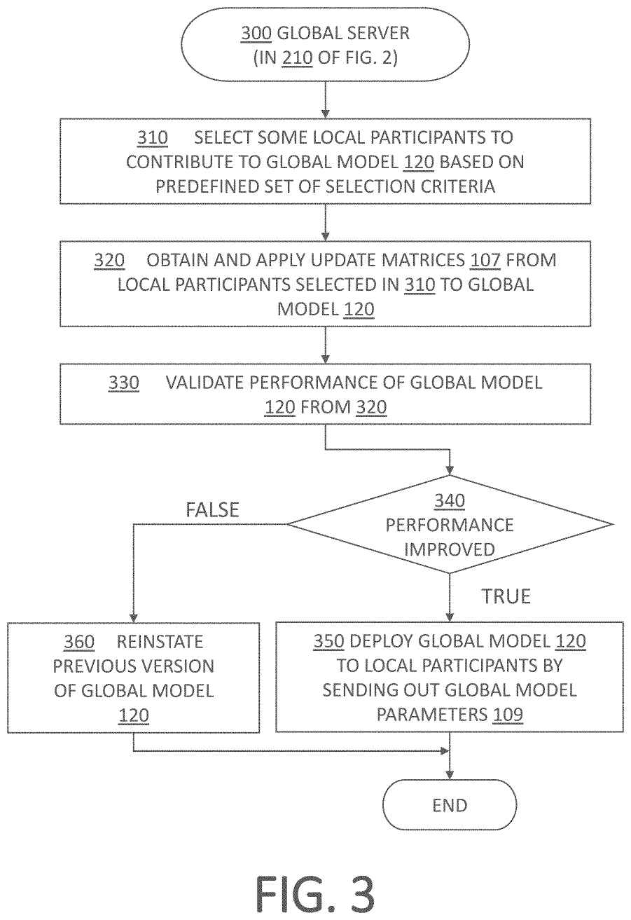

In block 210, the global server 110, by use of one or more computing platforms such as workstations or mainframes, performs functionalities including: training the global model 120 by using known optimization techniques and loss functions to minimize any loss function and to update parameters and corresponding weights of the global model 120; receiving metadata on the local dataset 170 including the size of the local dataset 170, demographics on patients of the local participants 150, and annotation quality metrics for the image data in the local dataset 170; receiving the update matrix 107 from the local participant 150; and updating and deploying the global model 120 to the local participant 150 by sending the global model parameters 109. Detailed operations in a learning cycle of the global model 120 are presented in FIG. 3 and corresponding description.

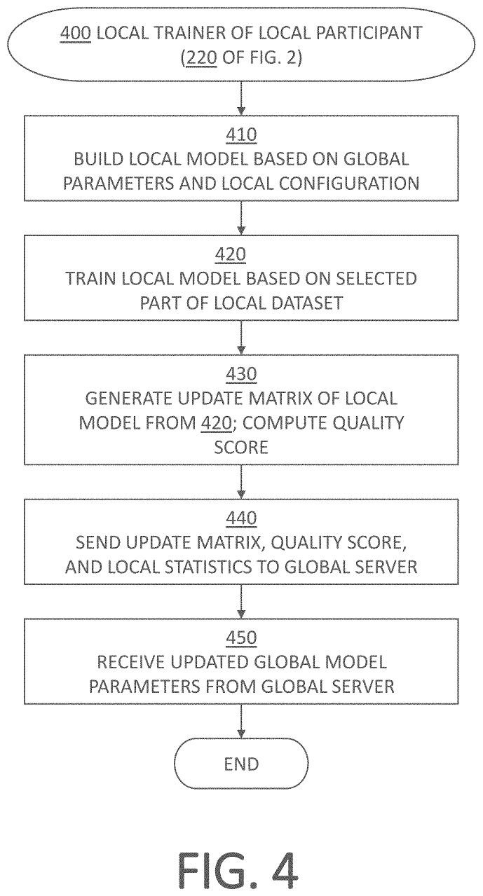

In block 220, the local participant 150, by use of one or more computing platforms such as workstations that are operatively coupled to imaging equipment in the hybrid OR 160, perform functionalities including: storing the local model 180 that is a replica of the global model 120 as released to all local participants of the federated learning system 100 or a variation of the global model 120 specific to the local participant 150 based on previous version of the global model parameters 109 and locally trained thereafter; training the local model 180 based on selected datapoints from the local dataset 170 and generating the update matrix 107, which includes either aggregated gradients based on the selected datapoints or updated values of the selected datapoints corresponding to parameters in the local model 180; encoding and/or encrypting the update matrix 107 for transmission to the global server 110; sending the update matrix 107 and local statistics from the local dataset 180 not parameterized in the local model 180 to the global server 110; sending and/or receiving the quality score 105 of the local participant 150 to and from the global server 110; and receiving the global model parameters 109 of the global model 120 from the global server 110 as updated after retraining of the global model 120 based on the update matrix 107 from all local participants from a previous learning cycle. Detailed operations in a learning cycle of the local model 180 are presented in FIG. 4 and corresponding description.

In certain embodiments, the local participant 150 participates in the federated learning by training the local model 180 but avoid learning from other participants such that the local participant 150 would keep the local model 180 as accurate as possible specifically for the local participant 150. In certain other embodiments, the local participants 150 participate in the federated learning by training the global model 120 in collaboration with other participants and by sharing the global model 120 amongst all participants such that the performance of the global model 120 would be improved on average across the participants. In certain other embodiments, the local participants 150 train the local models 180 respectively and directly learn from other local models 180 within a group of the local participants agreed to form a partnership.

FIG. 3 depicts a detailed workflow 300 of the global server 110 in a round of training for the global model 120.

Blocks 310 through 360 represent operations performed by the global server 110 in a round of training for the global model 120, amongst all operations performed by the global server 110 as represented in block 210 of FIG. 2. The global server 110 can be configured to perform a learning cycle as presented herein in a certain preconfigured interval, upon receiving a certain number of update matrices from all local participants, upon receiving a certain number of update matrices from a selected group of local participants based on a preconfigured condition, upon receiving a certain number of update matrices that represents changes outside of threshold ranges, or any combinations thereof.

In addition to the learning cycle shown in blocks 310 through 360, the global server 110 also performs functions including, but not limited to, coordinating a federated learning within the federated learning system 100 by communicating configurations of the federated learning such as hyperparameters for local models to local participants, producing individual performance or group performance reports as cumulated in the global dataset and/or represented in the global model, and sending the performance reports to respectively designated recipients amongst all of the local participants. The global server 110 can also propagate any changes in the quality scores 105 of the local participants 150 above a preconfigured threshold to other local participants. The global server 110, in concert with the local participant 150, can produce many types and levels of performance reports including differentiated performance information according to the recipient and share the performance reports accordingly from an individual to the local participants in the federated learning system 100.

The global server 110 can perform such tasks on coordination of the federated learning and performance reporting independently from and concurrently with the learning cycle shown in blocks 310 through 360. The hyperparameters of the federated learning system 100 indicates parameters of which values are used to control the learning process of the federated learning system such as a learning rate, regularization methods, weight decay and augmentation methods for learning by the local models 180.

In block 310, the global server 110 selects a subset of local participants that would contribute to training the global model 120 in a current learning cycle based on a predefined set of selection criteria. Then, the global server 110 proceeds with block 320.

In certain embodiments, the global server 110 selects the subset of local participants 150 to perform the learning cycle of the global model 120 for a proper representation of the local participants without wasting computing resources and network bandwidth for processing the local participants in the entirety. Based on the number of the local participants 150 in the federated learning system 100 and the respective characteristics of each of the local participants 150, all local participants 150 can be represented in the global model 120 by a properly selected subset from the local participants 150. In selecting the subset of the local participants 150 to be represented in the global model 120, the global server 110 examines, respective for all local participants, aspects including, but not limited to, availability/access schedule of the local participant 150, the quality score of the local participant 150, a mode of subscription of the federated learning system 100 by the local participant 150, and the quality of data generated from the local participant 150.

In selecting the local participants in block 310, the availability or an access schedule of the local participant 150 is considered to distribute workload on the computing platforms of the local participant 150 such that computations performed for the federated learning would not burden regular workload of the computing platforms of the local participant 150 in processing the image data generated from the hybrid OR 160 to facilitate procedures and medical functionalities for which the local participant 150 is responsible. Accordingly, the global server 110 would select local participants that currently have enough computational resources available for the federated learning system 100 for the subset in block 310.

In selecting the local participants in block 310, the quality score 105 of the local participant 150 represents the level of contribution to be made by the update matrix 107 of the local participant 150 to the global model 120, as noted above. Accordingly, the global server 110 selects local participants for the subset such that a distribution of the quality scores amongst the local participants in the subset is substantially similar to the distribution of the quality scores amongst all local participants.

In selecting the local participants in block 310, the mode of subscription by the local participant 150 in the federated learning system 100 configures the manner of participation by the local participant 150 in the federated learning, types of information exchanged, and entities in the federated learning system 100 communicating with the local participant 150. For example, the local participant 150 may subscribe to the federated learning system 100 in a mode which the local participant 150 would receive the global model parameters 109 as weighted in the global model 120 for building and updating the local model 170 but may opt out from contributing to the global model 120 and would not send the updates matrices 107 to the global server 110. Accordingly, the global server 110 would exclude any local participants subscribing in no-contribution mode from the subset for the current learning cycle for the global model 120.

In selecting the local participants in block 310, the global server 110 attempts to select the local participants 150 such that performance of the global model 120 would be improved by the current learning cycle. Accordingly, it is not desirable for the subset in block 310 to have any local participants 150 that has respective local datasets of poor quality which are, for example, images acquired on older systems and, therefore, of poor quality or containing noise. To screen data quality from the local participants, the global server 110 can hold out a subset of the global dataset 130 for validation, referred to as a validation dataset, apply the update matrices 107 from the local participants 150 to the global model 120 and test the global model 120 on the validation dataset, and select any local participants that had improved outcome of the global model 120 on the validation dataset for the subset for the learning cycle of the global model 120. If the outcome of the global model 120 had not been improved when tested with the validation dataset after being updated by the update matrices 107 from the local participants 150, then the global server 110 would reinstate the global model 120 to the state prior to applying the update matrix 107 from the local participant 150 and discard the local participant 150 from selection for the subset.

In certain embodiments, the global server 120 is configured to maintain the global model 120 without retraining in cases where the global server 120 had not discovered any local participants for the subset in block 310 according to the selection criteria as noted above.

In block 320, the global server 110 obtains the update matrix 107 from each of the local participants 150 selected from block 310 and applies the update matrices 107 collectively to the global model 120. The global model 120 would have weights for the global model parameters 109 respectively updated after the global server 110 completes block 320. Then, the global server 110 proceeds with block 330.

In certain embodiments, for an optimized neural network used for the local model 180, a weight of parameter X in the local model 180 is formulated as (wX+b) at each neural network layer in each of the local participants, where X represents the features learned by the neural network, w represents weights respective to the parameters, and b represent biases indicating a noise or a marginal error in representing the feature by the formula (wX+b). With a non-linear activation function σ, the weight in each of the local participants is computed as σ(wX+b).

In the same embodiments as above, when the weights and the biases are computed as noted above respectively at the plurality of local participants 150 in the federated learning system 100, the global server 110 can aggregate all weights for parameter X from respective local participants 150 in the global model 120 as σ((w_{A}+w_{B}+ . . . )X+(b_{A}+b_{B}+ . . . )), where w_{P} indicates a weight at local participant P, and b_{P} indicates a bias at local participant P, provided that the weights from the respective local participants are even in the respective quality scores. If the quality scores for respective local participants differ from one another, the global server 110 aggregates all weights for parameter X in training for the global model 110 based on: σ((q_{A}w_{A}+q_{B}w_{B}+ . . . )X+(q_{A}b_{A}+q_{B}b_{B}+ . . . )), where q_{P} indicates a quality score assigned to local participant P. Once a weight for parameter X in the global model 120 is updated according to either one of the update aggregation methods above, a new weight for parameter X is sent back to the local participant 150 as a part in the global model parameters 109 such that the new weight for parameter X is applied to the local model 180 in a next learning cycle based on the local dataset 170.

In the same embodiments as above, the global server 110 first generates a weighted average of the update matrices from the subset of local participants selected in block 310 by gradient aggregation learning algorithm and updates the global model 120 with the weighted average of the update matrices. The weighted average of the updated matrices in the global model 120 is obtained by dividing one of the aggregated weights computed as above corresponding to status of the quality scores of the local participant by the total number of local participants in the federated learning system 100. The global server 110 obtains the update matrices 107 as being respectively optimized by the local participant 150 on the local model 180 for the local dataset 170.

In block 330, the global server 110 validates the performance of the global model 120 updated from block 320. The global server 110 runs the global model 120 with a validation dataset set aside from the global dataset 130 that had not been used in training of the global model 120. In certain embodiments, the validation dataset of the global server 110 can be annotated by expert users or expert systems. Then, the global server 110 proceeds with block 340.

In block 340, the global server 110 determines whether or not the global model 120 as updated from block 320 performs better than a previous version of the global model 120 prior to the update from block 320. If the global server 110 determines that the performance of the global model 120 updated from block 320 has been improved over the previous version of the global model 120, then, the global server 110 proceeds with block 350. If the global server 110 determines that the performance of the global model 120 updated from block 320 had not been improved over the previous version of the global model 120, then, the global server 110 proceeds with block 360.

In block 350, the global server 110 deploys the global model 120 as updated from block 320 by sending out the global model parameters 109 to the local participants 150 of the federated learning system 100. The global model parameters 109 indicate the weighted parameters of the global model 120 as updated from block 320. The global server 110 sends the global model parameters 109 such that each of the local participants 150 can selectively utilize the global model parameters 109 for the local model 180 of the local participant 150. Then, the global server 110 terminates the current learning cycle for the global model 120.

In certain embodiments, the global server 110 may have a predetermined performance threshold, for example, a performance metric such as reliability of prediction of a previous version of the global model 120 prior to updating in block 320. In case where the global model 120 does not perform as well as the previous version of the global model 120, the global server 110 can reinstate the previous version of the global model 120 and terminate a current learning cycle.

In block 360, the global server 110 reinstates the previous version of the global model 120 and terminate the current learning cycle for the global model 120 as the update from block 320 had not improved the performance of the global model 120.

In certain embodiments, the global model 120 is trained to predict how long a mechanical thrombectomy would take by training with fluoroscopy image sequences. A mechanical thrombectomy, also referred to as a thrombectomy, is an interventional procedure of removing a blood clot from a blood vessel, commonly performed in the cerebral arteries, to treat an acute ischemic stroke. The mechanical thrombectomy is known to be a procedure with many challenges that requires expertise and years of experiences to perform properly and safely. Further, the duration of mechanical thrombectomies is known to be a significant indicator on post-procedure prognosis including the rate of recovery or chances of neurological disabilities and/or any postprocedural hemorrhage. Accordingly, the global model 120 is calibrated such that contributions from local model parameters weigh more for criteria specifying relevant expertise and experiences of an operating surgeon and a medical team attending the procedure than other criteria, in predicting the duration of the mechanical thrombectomy.

In the same embodiments as above, the global server 110 has the global model 120 with global model parameters updated by local model parameters weighted by attributes of criteria as configured for the federated learning system 100, for example, based on a quality score. Respective weights for the various criteria are calibrated to account expertise and years of experiences of an operating surgeon in mechanical thrombectomies more than other attributes in predicting the procedure duration of the thrombectomies, as noted above. The global server 110, then, deploys the global model 120 to the local participant 150 and all other local participants by sending the global model parameters 109 with corresponding weights as updated.

In the same embodiments as above, the local participant 150 represents Hospital A. The local participant 150 incorporates the global model parameters 109 received from the global server 110 to the local model 180, trains the local model 180 with the local dataset 170, and generates the update matrix 107 of Hospital H, denoted as ΔU_{A}. The local participant 150 subsequently sends to the global server 110 the update matrix 107 of Hospital A (ΔU_{A}) that had been generated as well as statistics of the local dataset 170 (S_{A}) used in generating the update matrix 107, but not image data in the local dataset 170.

In the same embodiments as above, the global server 110 compares the statistics of the local dataset 170 (S_{A}) with the statistics of the data as stored in the global dataset 130. The global server 110 may discover that, for example, the number of thrombectomies performed each week is twice as greater in the global dataset 130 than in the statistics of the local dataset 170 (S_{A}). The global server 110 may further discover that an average duration of thrombectomies is 30% shorter in the global dataset 130 than in the is statistics of the local dataset 170 (S_{A}). As noted above, because the average duration of thrombectomies is a significant indicator of a successful procedure regarding the recovery of patients, the global server 110 weighs the criteria affecting the duration of the procedure more than other criteria in the updates from the local model to the global model 120. Accordingly, the global server 120 may conclude that the local participant 150 of Hospital A is less skilled than average in mechanical thrombectomies compared to data in the global dataset of the federated learning system 100 coupled to the global server 120. The global server 120 consequently reduces a contribution of the update matrix 107 of Hospital A (ΔU_{A}) to the global model 120 by decreasing a quality score 105 corresponding to Hospital A.

The aforementioned embodiments illustrate how differences in global statistics and local statistics can affect the quality scores of the local participant 150, and consequently, a level of contribution of the local model 180 to the global model 120, by way of one-to-one interaction between the global server 110 and the local participant 150, Hospital A. It would be noted that each of the local participants 150 in the federated learning system 100 that is of the same mode of subscription would independently and concurrently interact with the global server 110 in a manner similar to the example of Hospital A as noted above.

FIG. 4 depicts a detailed workflow 400 of a local trainer of the local participant 150 in a round of training for the local model 180.

Blocks 410 through 450 represent a learning cycle of the local model 180 by the computing platform(s) of the local participant 150, which is referred to as a local trainer herein, as being distinguished from additional roles in the context of the federated learning performed by the local participant 150.

In block 410, the local trainer builds the local model 180 by use of the global model parameters 109 that had been previously obtained and local model configurations specific to the local participant 150. In certain embodiments where no local model configurations have been specified, the local trainer takes the global model parameters 109 as provided from the global server 110 for the local model 180. Then, the local trainer proceeds with block 420.

The local model 180 used in each learning cycle may differ from one cycle to the next, based on the local model configuration. The local model 180 can be the same as the global model 120 as previously deployed by the global model parameters 109 in an immediately preceding learning cycle of the global model 120, in cases where the local model configuration specified that the local model 180 to be the same as the global model 120 from the latest global model learning cycle. The local model configuration can also set the local model 180 to be any version, released or made available to the local trainer without deployment, of the global model 120 or a variation of the global model 120 that incorporate any aspects specific for the local participant 150.

In block 420, the local trainer trains the local model 180 as built from block 410 with selected part of the local dataset 170, including image data from the hybrid OR 160 and local statistics. The local trainer also computes the quality score 105 for the local participant 150 based on the local dataset 170 and the local statistics of the local participant 150. The local trainer can select, for example, any data in the local dataset 170 including data that are timestamped after the latest learning cycle for the local model 180. The local trainer may also perform a local validation on a local held out validation dataset. In case where the local model does not perform as well as the previous version of the local model, the local server can reinstate the previous version of the local model and terminate a current learning cycle. In the case where the local model outperforms the previous version of the local model, the local trainer proceeds with block 430.

In block 430, the local trainer generates the update matrix 107 of the local model 120 from block 420. The update matrix 107 indicates a collective difference in weights between respective parameters of the local model 180 before and after a learning cycle based on the local dataset 170, as noted in FIG. 1. Accordingly, the update matrix 107 represents the collective difference either as an aggregate gradient computed on multiple subsets of the local dataset 170 or as updated weight values for a set of parameters of the local model 180. The local trainer can encode, encrypt, or encode and encrypt the update matrix 107 for secure transmission to the global server 110. Then, the local trainer proceeds with block 440.

In certain embodiments, the local trainer computes a gradient of the loss using stochastic gradient descent (SGD) optimization method over all or small batch of training instances from the local dataset 170 generated and stored in the local participant 150. The SGD, a version of gradient descent (GD), is an iterative first-order optimization algorithm used to find a local minimum or maximum of a given function, which is commonly used in machine-learning (ML) and deep learning (DL) to minimize a cost or loss function σs in a linear regression. The amount of overall computational cost of the local learning can be reduced and managed within a predefined limit by selecting a certain fraction of the local participants with sufficient computing resources to perform optimization on each learning cycle, and/or by setting sizes of minibatches selected from the local dataset 170 for the local learning cycles.

The hyperparameters utilized in the local participant 150 and the values of hyperparameters are selected and instantiated by the global server 110. If several training instances are used in a single learning cycle, the local trainer computes an aggregated gradient of the training instances at the local participant as the local model 170 after or intermittently during the learning cycle.

In the same embodiments as in block 320 of FIG. 3, in which the weight of parameter X in the local model 180 is computed as σ(wX+b) in each of the local participants, the update matrix 107 for the local participant 150 would be ΔU_{A}=σ(wX+b)_{T1}−σ(wX+b)_{T2}, where T1 represent a point of time prior to a current local learning cycle, and T2 represent a point of time subsequent to the current local learning cycle.

In block 440, the local trainer sends the update matrix 107, the quality score 105, and local statistics to the global server 110. Similar to the update matrix 107, the local trainer can encode and/or encrypt the quality score 105 and the local statistics for the transmission. The local statistics is a part of the local dataset 170 that can be transmitted to the global server 110 as no sensitive information regarding patient privacy is represented in the local statistics. Then, the local trainer proceeds with block 450.

In block 450, the local trainer receives a new set of the global model parameters 109, as the global model 120 had been updated, from block 320 of FIG. 3, by incorporating the update matrix 107 based on the quality score 105 that had been sent in block 440. The global model parameters 109 received in block 450 of the local trainer is responsive to sending of the global model parameters by the global server 110 in block 350 of FIG. 3. The local trainer can also receive quality scores 105 of other local participants as propagated by the global server 110 as well as the global statistics in the global model learning cycle shown in FIG. 3. The local trainer would utilize the new set of the global model parameters 109 received in block 450 to deploy for use or to build or adapt the local model 180 for a next local learning cycle. Then, the local trainer concludes the current local learning cycle.

It should be noted that the local trainer and the global server 110 share responsibility in optimization of the global model 120. For example, if the local participant 150, Hospital A, performs, on average, half as many procedures in the hybrid OR 160 as the number of procedures shown from the global statistics from the global server 110, a linear scaling can be applied to give half as much weight to the update matrix 107, denoted as ΔU_{A}, for the local participant 150 of Hospital A. Other non-linear scaling can also be applied based on other aspects of the local participant 150. Further, the local trainer would also adjust the quality score 105 for the local participant 150, Hospital A such that the global server 110 can further optimize the scaling of the update matrix 107 by iteratively reducing the weights applied to the update matrix 107 (ΔU_{A}) while incorporating the update matrix 107 to the global model 120. Accordingly, the global server 110 can prevent the performance of the global model 120 on a validation dataset from being reduced as compared to a version of the global model 120 before incorporating the update matrix 107 (ΔU_{A}).

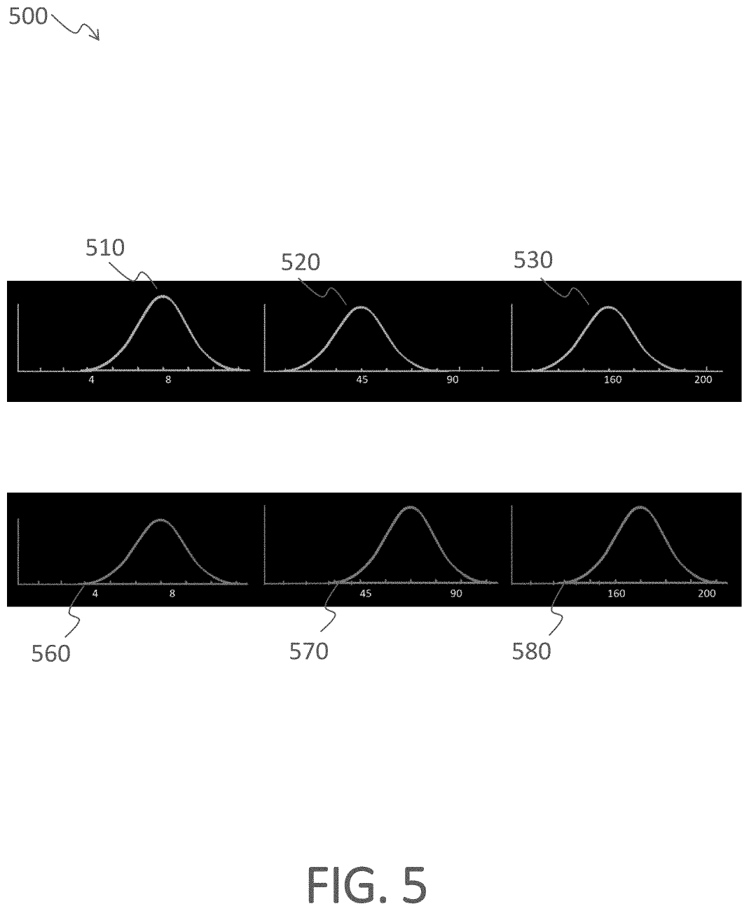

FIG. 5 illustrates exemplary graphs 500 from the local statistics and the global statistics on a few parameters of the global model 120 for predicting a duration of navigation to a surgery site in mechanical thrombectomy.

As noted in the example of the global model 120 for predicting the duration of mechanical thrombectomy in FIG. 3, the global model 120 (which can contain multiple global models trained for different tasks) can be used for predicting the duration of navigation to the surgery site, often the cerebral arteries in the head of the patient, as the duration of navigation to the surgery site takes the significant amount of time in the duration of the mechanical thrombectomy in its entirety. It should be noted that the surgical tools may be inserted at the femoral artery at the groin in the mechanical thrombectomy, and accordingly, the length of the vasculature the surgical tools must navigate through is the entire torso, through the aorta, the neck, and the head. The global model 120 contains updates from local model parameters weighted by, for example, quality scores from two criteria of the number of procedures per week and the height of the patient undergoing the procedure for predicting the duration of the navigation. Graphs 510, 520, and 530 represent the global statistics. Graphs 560, 570, and 580 represent the local statistics.

A graph on the number of procedures per week in the global statistics 510 and a graph on the number of procedures per week in the local statistics 560 show substantially similar frequencies and distributions about an average of eight (8) times per week. Accordingly, the local participant 150 contributing to the local statistics is regarded as being equal to the global dataset 130 contributing to the global statistics in expertise and experiences.

A graph on the duration of navigation to the surgery site in the global statistics 520 and a graph on the duration of navigation to the surgery site in the local statistics 570, however, show a significant difference in the respective durations of navigation. The global statistics in graph 520 shows that the duration is on average 45 minutes and the local statistics in graph 570 shows that the duration is on average 68 minutes.

A graph on the height of the patient in the global statistics 530 and a graph on the height of the patient in the local statistics 580 show patterns similar to the duration of navigation. The global statistics in graph 530 shows that the height of the patient is on average 160 centimeters (cm), or 5′3″, and the local statistics in graph 580 shows that the height of the patient is on average 170 cm, or 5′7″.

As noted in the example of FIG. 3, a longer duration of the mechanical thrombectomy than the global statistics may indicate a lower level of expertise or a lack of experience on the local participant, even if the respective numbers of procedures per week in the local statistics and the global statistics are substantially similar. However, a longer duration of navigation can be logically inferred as caused by a longer distance to navigate, and the height of the patient can be hypothetically identified as affecting the duration of navigation. The local statistics in graph 580 actually show that the patients at the local participant were on average, 10 cm, or 4 inches, taller than the patients represented in graph 530 of the global statistics. Accordingly, the criterium of the height of the patient is confirmed to be a factor in predicting the duration of the navigation by the global model 120, and the quality score computed for the local participant is not degraded due to the longer duration of navigation.

In another example, the global model 120 (which can contain multiple global models trained for different tasks) for predicting the duration of navigation can assign respective weights accordingly by analyzing image data, for example, fluoroscopy sequences, from the local dataset 180. In this example, the local statistics and the global statistics for the criteria of the number of procedures per week and the height of the patient are substantially similar, but there still are statistically meaningful differences in the duration of navigation as shown in graphs 520 and 570. Amongst many likely factors affecting the duration of navigation, the shapes of the blood vessels through which the surgical tools must navigate, referred to as vascular anatomy, is analyzed because it is known in the medical knowledgebase that the shapes of blood vessels of patients affect easiness, and accordingly, the duration, of the navigation.

Although the vascular anatomy of the patients should be analyzed and evaluated to determine if and how the vascular anatomy of the patient affects the prediction of the duration of the navigation by the global model 120, the image data cannot be uploaded to the global server 110 for protection of the sensitive data and patient privacy. Accordingly, the local participant 150 would request to download the local learning tools 108 to analyze and evaluate image data stored in the local dataset 170. The local participant 150 may build a local image model separate from the local model 180 for predicting the duration of the navigation. Based on the image data from the local dataset 170 such as fluoroscopy sequences or digital subtraction angiography (DSA), the local participant can detect, for example, tortuosity in vasculature of the local patients from image data, indicating curvy blood vessels. To ensure the analysis and evaluation of the image data at respective local participants are performed uniformly across all of the local participants in the federated learning system 100, the local learning tools 108 are supported by the global server 110 rather than letting each local participant select any image analysis tools and programs from one local participant to another. The local learning tools 108 can be configured to send image statistics for features suspected to be a factor in the duration of the navigation, at the end of analyses and evaluations on the local image data. Exemplary aspects of the image statistics include, but are not limited to, a prevalence of tortuous vessel or numerical representation of tortuosity as in angles and a percentage of the vessels, and a prevalence of plaques in vasculature of the patients, which would have blocked the surgical tools from passing through. The global server 110 can collect local image statistics from all local participants and compare with global statistics to see if the local participant had the patients with more challenging vasculature for navigation to explain the longer duration of the navigation as noted above.

In still another example, the federated learning system 100 supports an image quality (IQ) processor to be installed in computing platforms in the hybrid ORs 160 of the local participants 150 such that image quality on each of the local participants 150 would be consistent across the federated learning system 100. For example, the IQ processor at one of the local participants 150, Institute B, analyzed image data in the local dataset 170 of Institute B and evaluates that Institute B generates the image data of lower resolution and with more noise than image data from other local participants or sample image data stored in the global dataset 130. Accordingly, the global server 110 would determine that annotations on the image data by Institute B would not be as accurate as annotations by other local participants and that training the global model 120 with weights from Institute B on parameters based on image analysis and evaluation would degrade performance of the global model 120 in predicting the duration of navigation, provided that high level statistics from Institute B are substantially similar to other local participants or to the global dataset 130. Based on the sub-par image quality of Institute B, the global server 110 can reduce the influence of local model weights from Institute B, denoted as w_{B} and b_{B}, by applying a lower quality score to Institute B, denoted as q_{B}. An application supporting analyses and evaluations of local image data by the IQ processor may be available for the local participants as well from the local learning tools 108 of the global server 110. It should be noted that Institute B can be assigned to a new quality score for local models trained to perform tasks other than predicting for the duration of navigation in mechanical thrombectomy, and that the global server 110 evaluates weights from each local participant and balances quality scores from each local participant per model and/or per task.

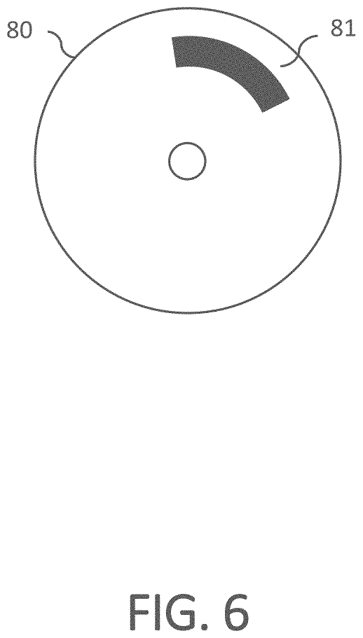

FIG. 6 shows an optical disc 80 as an example of a computer-readable medium comprising data.

The methods operative in the federated learning system 100 may be implemented on a computer as a computer-implemented method, as dedicated hardware, as firmware, or as a combination thereof. As also illustrated in FIG. 6, instructions for the computer, e.g., executable code, may be stored on a computer readable medium 80, e.g., in the form of a series 81 of machine-readable physical marks and/or as a series of elements having different electrical, e.g., magnetic, or optical properties or values. The medium 80 may be transitory or non-transitory. Examples of computer readable mediums include memory devices, optical storage devices, integrated circuits, servers, online software, etc.

Examples, embodiments or optional features, whether indicated as non-limiting or not, are not to be understood as limiting the present disclosure as claimed.

While the present disclosure has been illustrated and described in detail in the drawings and foregoing description, such illustration and description are to be considered illustrative or exemplary and not restrictive; the present disclosure is not limited to the disclosed embodiments.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A system for machine-learning in a hybrid operating room environment, the system comprising:
a global server comprising one or more processors configured to:
select a local participant subset from a plurality of local participants associated with a hybrid operating room;
subscribe the local participant subset to a federated learning system to contribute to a global machine-learning model configured to perform a task in the hybrid operating room;
obtain an update matrix and a quality score from each local participant in the local participant subset;
update the global machine-learning model based on the update matrix and the quality score from each local participant in the local participant subset; and
deploy parameters and parameter weights of the global machine-learning model to each local participant in the local participant subset for building a local machine-learning model configured to perform the task in the hybrid operating room.

2. The system of claim 1, wherein the one or more processors are further configured to:
ascertain that the updated global machine-learning model needs to be validated prior to the deployment to each local participant in the federated learning system; and
validate that the updated global machine-learning model performs better than a pre-updated version of the global machine-learning model, by use of a validation dataset stored in the global server from which the global machine-learning model is trained.

3. The system of claim 1, wherein the local participant subset is selected based on a predefined selection criteria comprising at least one of: an availability schedule for each local participant in the federated learning system, the quality score for each local participant in the federated learning system, a mode of subscription to the federated learning system by each local participant in the federated learning system, and a quality of data generated from each local participant in the federated learning system.

4. The system of claim 1, wherein, to update the global machine-learning model, the one or more processors are further configured to:
aggregate weights of parameters in the update matrix from each local participant based on the respective quality score from each local participant; and
incorporate the aggregated weights of the parameters in the update matrix into parameter weights of corresponding parameters in the global machine-learning model.

5. The system of claim 1, wherein to deploy the parameters of the global machine-learning model, the one or more processors are further configured to:
send the parameters of the global machine-learning model to each local participant in the federated learning system.

6. The system of claim 1, wherein the one or more processors are further configured to:
obtain local statistics on predetermined aspects of each local participant in the local participant subset;
analyze correlations between the predetermined aspects and a task of the global machine-learning model as represented by differences in the local statistics and global statistics on the predetermined aspects in the federated learning system; and
adjust the parameter weights of the parameters in the global machine-learning model based on the correlations.

7. The system of claim 1, wherein the global machine-learning model comprises weighting of local machine-learning model parameter contributions to the parameters of the global machine-learning model according to one or more of: institute criteria on organizational characteristics of each local participant; imaging system criteria based on technical specifications of imaging systems in the hybrid operating rooms of each local participant; procedure criteria based on characteristics of procedures performed in the hybrid operating rooms; healthcare processional criteria based on characteristics of medical personnel working for each local participant; and patient criteria based on characteristics of patients receiving the procedures performed in the hybrid operating rooms.

8. The system of claim 1, further comprising:
a local client of a local participant in the local participant subset, the local client comprising one or more local processors configured to:
build the local machine-learning model configured to perform the task in the hybrid operating room of the local participant based on the parameters and the parameter weights of the deployed global machine-learning model, wherein the local participant includes a local dataset with image data and parameters associated with medical imaging equipment of the hybrid operating room of the local participant;
train the local machine-learning model with selected instances from the local dataset and local statistics of the local participant;
generate an update matrix representing a collection of respective differences in weights between respective parameters of the local machine-learning model before and after a learning cycle based on the local dataset;
send, to the global server, the update matrix, the quality score, and the local statistics for the local participant;
receive the parameters and the parameter weights of the global machine-learning model updated based on the update matrix, the quality score, and the local statistics sent from each local participant in the federated learning system; and
update the local machine-learning model for the local participant based on the updated parameters and the updated parameter weights of the global machine-learning model.

9. The system of claim 8, wherein the one or more local processors are further configured to:
compute the quality score of the local participant to indicate a level of contribution to the global machine-learning model by the local participant based on the local dataset.

10. The system of claim 8, wherein the local statistics stored in the local dataset comprises historical data generated from or observed at the local participant.

11. The system of claim 8, wherein the one or more local processors are further configured to:
download one or more local learning tools from the global server;
analyze image data stored in the local dataset of the local participant by use of the one or more local learning tools; and
send statistics resulting from the analyzed image data to the global server with weights of features, from the image data, parametrized in the local machine-learning model, wherein the weights of the features are calculated based on contribution of the parametrized features from the image data to a task set configured to be performed by the local machine-learning model.

12. A computer-implemented method for machine-learning in a hybrid operating room environment, the method comprising:
selecting a local participant subset from a plurality of local participants associated with a hybrid operating room;
subscribing the local participant subset to a federated learning system in a global server to contribute to a global machine-learning model configured to perform a task in the hybrid operating room;
obtaining an update matrix and a quality score from each local participant in the local participant subset;
updating the global machine-learning model based on the update matrix and the quality score from each local participant in the local participant subset; and
deploying parameters and parameter weights of the global machine-learning model to each local participant in the local participant subset for building a local machine-learning model configured to perform the task in the hybrid operating room.

13. The method of claim 12, further comprising:
ascertaining that the updated global machine-learning model needs to be validated prior to the deployment to each local participant in the federated learning system; and
validating that the updated global machine-learning model performs better than a pre-updated version of the global machine-learning model, by use of a validation dataset stored in the global server from which the global machine-learning model is trained.

14. The method of claim 12, wherein the local participant subset is selected based on predefined selection criteria comprising at least one of: an availability schedule for each local participant in the federated learning system, the quality score for each local participant in the federated learning system, a mode of subscription to the federated learning system by each local participant in the federated learning system, and a quality of data generated from each local participant in the federated learning system.

15. The method of claim 12, wherein the updating of the global machine-learning model further comprises:
aggregating weights of parameters in the update matrix from each local participant based on the respective quality score from each local participant; and
incorporating the aggregated weights of the parameters in the update matrix into parameter weights of corresponding parameters in the global machine-learning model.

16. The method of claim 12, further comprising:
obtaining local statistics on predetermined aspects of each local participant in the local participant subset;
analyzing correlations between the predetermined aspects and a task of the global machine-learning model as represented by differences in the local statistics and global statistics on the predetermined aspects in the federated learning system; and
adjusting the parameter weights of the parameters in the global machine-learning model based on the correlations.

17. The method of claim 12, wherein the global machine-learning model comprises weighting of local machine-learning model parameter contributions to the parameters of the global machine-learning model according to one or more of: institute criteria on organizational characteristics of each local participant; imaging system criteria based on technical specifications of imaging systems in the hybrid operating rooms of each local participant; procedure criteria based on characteristics of procedures performed in the hybrid operating rooms; healthcare processional criteria based on characteristics of medical personnel working for each local participant; and patient criteria based on characteristics of patients receiving the procedures performed in the hybrid operating rooms.

18. The method of claim 12, further comprising:
building the local machine-learning model configured to perform the task in the hybrid operating room of a local participant based on the parameters and the parameter weights of the deployed global machine-learning model, wherein the local participant includes a local dataset with image data and parameters associated with medical imaging equipment of the hybrid operating room of the local participant;
training the local machine-learning model with selected instances from the local dataset and local statistics of the local participant;
generating an update matrix representing a collection of respective differences in weights between respective parameters of the local machine-learning model of the local participant before and after a learning cycle based on the local dataset;
sending, to the global server, the update matrix, a quality score, and the local statistics for the local participant;
receiving the parameters and the parameter weights of the global machine-learning model updated based on the update matrix, the quality score, and the local statistics sent from each local participant in the federated learning system; and
updating the local machine-learning model for the local participant based on the updated parameters and the updated parameter weights of the global machine-learning model.

19. The method of claim 18, further comprising:
computing the quality score of the local participant to indicate a level of contribution to the global machine-learning model by the local participant based on the local dataset.

20. The method of claim 18, further comprising:
downloading one or more local learning tools from the global server;
analyzing image data stored in the local dataset of the local participant by use of the one or more local learning tools; and
sending statistics resulting from the analyzed image data to the global server with weights of features, from the image data, parametrized in the local machine-learning model, wherein the weights of the features are calculated based on contribution of the parametrized features from the image data to a task set configured to be performed by the local machine-learning model.
